(19) United States
(12) Patent Application Publication
(10) Pub. No.: US 2016/0287453 A1
(43) Pub. Date: Oct. 6, 2016

(54) Compression Bandage

(71) Applicant: JBC Corp, Virginia Beach, VA (US)

(72) Inventor: Brian Cavolt, Virginia Beach, VA (US)

(21) Appl. No.: 15/090,014

(22) Filed: Apr. 4, 2016

Related U.S. Application Data
(60) Provisional application No. 62/141,920, filed on Apr. 2, 2015.

(51) Int. Cl. A61F 13/64; A61F 13/62; A61F 13/02; A61F 13/56

(52) U.S. Cl. CPC A61F 13/64; A61F 13/56; A61F 13/622; A61F 13/0273; A61F 2013/5672

(57) ABSTRACT

A bandage with a raised retention element that generates a compressive force on a wound is provided. The bandage generally comprises a raised retention element disposed on an inner surface of a stretchable material. A pad may also be included on the inner surface of the stretchable material, and the pad may be placed over a wound. Then, a user may wrap the bandage around a portion of the injured person's body such that the stretchable material stretches and wraps upon itself. The raised retention element on the inner surface of the stretchable material has a higher coefficient of friction than the stretchable material, and thus, the raised retention element helps secure the bandage around the portion of the injured person's body to generate a compressive force on the wound.

### CROSS-REFERENCE TO RELATED APPLICATIONS

This application claims priority under 35 U.S.C. §119(e) to U.S. Provisional Patent Application Ser. No. 62/141,920 filed Apr. 2, 2015, which is incorporated herein in its entirety by reference.

### FIELD OF THE INVENTION

The invention generally relates to compression bandages for treating injuries, and more specifically, to compression bandages that retain the bandage in a predetermined location.

### BACKGROUND OF THE INVENTION

Medical supplies are necessary for treating injuries sustained by military personnel, hikers, construction workers, loggers, and other persons subject to bodily injury. Depending on the type of injury, the treatment protocol may call for immediate pressure to a wound, for example, to stem the flow of blood from the wound.

Prior art bandages rely on numerous components to apply pressure to a wound. Examples of these prior art device may be found in U.S. Pat. Nos. 5,628,723, 6,593,508, 7,490,634, and 7,600,540; U.S. Patent Publication No. 2011/0092873; and European Patent No. 0957771, all of which are incorporated by reference herein in their entirety.

These bandages have several components such as hooks or knobs to help secure the bandage over a wound. The additional components add complexity to the bandages, and thus, more opportunities for failure and increased production costs. Further yet, the increased complexity slows the ability of a person to quickly apply pressure to the wound. Therefore, there is a need for an improved bandage solution that provides effective treatment, reduces production costs, and assures proper retention of the bandage in a predetermined location.

### SUMMARY OF THE INVENTION

It is thus one aspect of embodiments of the present invention to provide a bandage with an improved design that can generate a compressive force on a wound. Specifically, it is an aspect of embodiments of the invention to provide a compression bandage with a raised retention element to help provide the compressive force on the wound, for example, to stem the flow of blood from the wound.

It is another aspect of embodiments of the invention to provide a compressive bandage with various configurations of raised retention elements to retain the bandage in a predetermined location and to provide a compressive force on a wound. In some embodiments, the bandage employs a plurality of raised retention elements extending between a first end and a second end of the stretchable material of the bandage. However, it will be appreciated that the raised retention elements may have other orientations relative to the stretchable material of the bandage. Further, the number of raised retention elements may also vary. Exemplary embodiments having three raised retention elements are discussed herein, but embodiments of the invention may also include one, two, four, etc. raised retention elements.

It is another aspect of embodiments of the invention to provide a compressive bandage with a plurality of individual strands that combine to form a raised retention element. The individual strands may be held in place by weft threads, which are threads oriented in a lateral direction of the bandage or stretchable material. In some embodiments, the strands may simply be straight and oriented parallel with the warp threads, which are threads oriented in a longitudinal direction of the bandage or stretchable material. However, it will be appreciated that numerous strand configurations may be employed. For example, strands may number more or less than seven, and the strands may be oriented in different directions relative to the warp and weft directions and may form various geometric designs besides simply being straight.

It is yet another aspect of embodiments of the invention to provide a compressive bandage wherein a pad is interconnected to a stretchable material at a central location. The pad is placed over a wound, and the pad provides absorbability. Prior art pads are interconnected to a strap or material around a perimeter of the pad, which compresses the pad and reduces the absorbability of the pad. In contrast, the central interconnection of the invention allows a greater area of the pad to remain uncompressed, which increases the pads ability to absorb fluid. This configuration also allows the pad to move relative to the stretchable material and remain over the wound.

One particular embodiment of the invention is a compression bandage for treating an injury, comprising a stretchable material having a first end, a second end, and a first length between the first and the second ends when the stretchable material is in a relaxed state, the stretchable material having a second length between the first and second ends when the stretchable material is in a tensioned state, the second length being longer than the first length, the stretchable material also having a first side, a second side, and a first friction coefficient; a retention element disposed on the first side of the stretchable material, and the retention element raised above the first side, the raised retention element having a second friction coefficient, the second friction coefficient being greater than the first friction coefficient; a pad disposed on the first side of the stretchable material; and a securing fastener positioned proximate to the second end of the stretchable material, wherein the securing fastener is configured to hold the second end of the stretchable material in the tensioned state against the second side of the stretchable material such that the raised retention element contacts the second side of the stretchable material and prevents the second end of the stretchable material from slipping relative to the second side of the stretchable material.

Embodiments of the invention may comprise addition features. For example, in some embodiments the securing fastener is one of a hook and loop fastener, a button, a tape, a snap fastener, a latch, a magnet, an adhesive, a lug, a zipper, a force provided by a user, a suction cup, an electric induction, and a clip. In various embodiments, the raised retention element longitudinally extends between the first end and the second end of the stretchable material. In some embodiments, a second retention element is disposed on the first side of the stretchable material, and the second retention element is raised above the first side, the second raised retention element also having the second friction coefficient. In various embodiments, the raised retention elements longitudinally extend between the first end and the second end of the stretchable material, and the raised retention elements are oriented substantially parallel to each other.

In some embodiments, the pad is interconnected to the stretchable material at a central location of the pad. In various embodiments, the pad is positioned proximate to the first end of the stretchable material. In some embodiments, the raised retention element comprises a plurality of strands, and wherein each strand forms a non-zero angle with a longitudinal plane extending from the first end to the second end of the stretchable material. In various embodiments, the raised retention element comprises a plurality of strands, wherein each strand forms a sinusoidal pattern as each strand extends from the first end to the second end of the stretchable material.

Another particular embodiment of the invention is a method for securing a bandage to an appendage of a person, comprising (i) providing a stretchable material having a first end, a second end, a first side, a second side, and a first friction coefficient; (ii) providing a retention element on the first side of the stretchable material, the retention element is raised above the first side, the raised retention element having a second friction coefficient, the second friction coefficient being greater than the first friction coefficient; (iii) providing a pad on the first side of the stretchable material; (iv) locating the pad on an appendage of a person; (v) wrapping the stretchable material around the appendage such that at least a portion of the raised retention element contacts the second side of the stretchable material to induce a friction force; and (vi) securing the portion of the raised retention element against the second side of the stretchable material with a securing fastener.

Embodiments of the invention may comprise addition features. For example, some embodiments comprise (vii) stretching the stretchable material between a first length and a second length to wrap the stretchable material around the appendage, wherein the second length is longer than the first length. In various embodiments, the method comprises (viii) providing the securing fastener, wherein the securing fastener is one of a hook and loop fastener, a button, a tape, a snap fastener, a latch, a magnet, an adhesive, a lug, a zipper, a force provided by a user, a suction cup, an electric induction, and a clip. In some embodiments, the raised retention element longitudinally extends between the first end and the second end of the stretchable material. In various embodiments, the method comprises (ix) providing a second retention element on the first side of the stretchable material, the second retention element is raised above the first side, the second raised retention element also having the second friction coefficient. In some embodiments, the method comprises (x) providing the raised retention elements, wherein the raised retention elements longitudinally extend between the first end and the second end of the stretchable material, and the raised retention elements are oriented substantially parallel to each other.

In various embodiments, the method comprises (xi) interconnecting the pad to the stretchable material at a central location of the pad. In some embodiments, the method comprises (xii) positioning the pad proximate to the first end of the stretchable material. In various embodiments, the method comprises (xiii) providing the raised retention element, wherein the raised retention element comprises a plurality of strands, and wherein each strand forms a non-zero angle with a longitudinal plane extending from the first end to the second end of the stretchable material. In various embodiments, the method comprises (xiv) providing the raised retention elements, wherein the raised retention element comprises a plurality of strands, wherein each strand forms a sinusoidal pattern as each strand extends from the first end to the second end of the stretchable material.

Yet another particular embodiment of the invention is a compression bandage system for treating an injury, comprising a stretchable material having a first end, a second end, and a first length between the first and the second ends when the stretchable material is in a relaxed state, the stretchable material having a second length between the first and second ends when the stretchable material is in a tensioned state, the second length being longer than the first length, the stretchable material also having a first side, a second side, and a first friction coefficient; a plurality of raised retention elements disposed on the first side of the stretchable material, wherein the raised retention elements longitudinally extend between the first end and the second end of the stretchable material, and the raised retention elements are oriented substantially parallel to each other, wherein the raised retention elements each comprise a plurality of strands, wherein each strand forms a sinusoidal pattern as each strand extends from the first end to the second end of the stretchable material, the raised retention elements having a second friction coefficient, the second friction coefficient being greater than the first friction coefficient; a pad disposed on the first side of the stretchable material at a central location of the pad, and the pad proximate to the first end of the stretchable material; and a securing fastener positioned proximate to the second end of the stretchable material, wherein the securing fastener holds the second end of the stretchable material in place relative to the second side of the stretchable material such that the plurality of raised retention elements contacts the second side of the stretchable material and prevents the second end of the stretchable material from slipping relative to the second side of the stretchable material.

These and other advantages will be apparent from the disclosure of the invention(s) contained herein. The above-described embodiments, objectives, and configurations are neither complete nor exhaustive. The Summary of the Invention is neither intended nor should it be construed as being representative of the full extent and scope of the invention. Moreover, references made herein to “the invention” or aspects thereof should be understood to mean certain embodiments of the invention and should not necessarily be construed as limiting all embodiments to a particular description. The invention is set forth in various levels of detail in the Summary of the Invention as well as in the attached drawings and Detailed Description and no limitation as to the scope of the invention is intended by either the inclusion or non-inclusion of elements, components, etc. in this Summary of the Invention. Additional aspects of the invention will become more readily apparent from the Detailed Description particularly when taken together with the drawings.

### BRIEF DESCRIPTION OF THE DRAWINGS

The accompanying drawings, which are incorporated in and constitute a part of the specification, illustrate embodiments of the disclosure and together with the general description of the disclosure given above and the detailed description of the drawings given below, serve to explain the principles of the disclosures.

FIG. 1A is a perspective view of a compression bandage in accordance with various embodiments of the invention;

FIG. 1B is a perspective view of a compression bandage in vacuum-sealed packaging in accordance with various embodiments of the invention;

FIG. 1C is a top plan view of a securing fastener in accordance with various embodiments of the invention

FIG. 2 is a top plan view of a compression bandage with multiple raised retention elements in accordance with various embodiments of the invention;

FIG. 3 is a detailed view of a raised retention element of a stretchable material in accordance with various embodiments of the invention;

FIG. 4 is a top plan view of a compression bandage with an offset strand configuration in accordance with various embodiments of the invention;

FIG. 5 is a top plan view of a compression bandage with a layered strand configuration in accordance with various embodiments of the invention;

FIG. 6 is a top plan view of a compression bandage and various dimensions in accordance with various embodiments of the invention; and

FIG. 7 is a diagram of a particular warp and weft combination in accordance with various embodiments of the invention.

To assist in the understanding of the embodiments of the invention the following list of components and associated numbering found in the drawings is provided herein:

| Component No. | Component |
|---|---|
| 2 | Compression Bandage |
| 4 | Stretchable Material |
| 6 | First Side |
| 8 | Second Side |
| 10 | Raised Retention Element |
| 12 | Pad |
| 14 | Securing Fastener |
| 16 | Packaging |
| 18 | First End |
| 20 | Second End |
| 22 | First Raised Retention Element |
| 24 | Second Raised Retention Element |
| 26 | Third Raised Retention Element |
| 28 | Central Location |
| 30 | Strand |
| 32 | Strand Angle |
| 34 | Overall Length |
| 36 | Gripping Width |
| 38 | Pad Length |
| 40 | Overall Width |

It should be understood that the drawings are not necessarily to scale, and various dimensions may be altered. In certain instances, details that are not necessary for an understanding of the invention or that render other details difficult to perceive may have been omitted. It should be understood, of course, that the invention is not necessarily limited to the particular embodiments illustrated herein.

### DETAILED DESCRIPTION

The invention has significant benefits across a broad spectrum of endeavors. It is the Applicant's intent that this specification and the claims appended hereto be accorded a breadth in keeping with the scope and spirit of the invention being disclosed despite what might appear to be limiting language imposed by the requirements of referring to the specific examples disclosed. To acquaint persons skilled in the pertinent arts most closely related to the invention, a preferred embodiment that illustrates the best mode now contemplated for putting the invention into practice is described herein by, and with reference to, the annexed drawings that form a part of the specification. The exemplary embodiment is described in detail without attempting to describe all of the various forms and modifications in which the invention might be embodied. As such, the embodiments described herein are illustrative, and as will become apparent to those skilled in the arts, and may be modified in numerous ways within the scope and spirit of the invention.

Although the following text sets forth a detailed description of numerous different embodiments, it should be understood that the detailed description is to be construed as exemplary only and does not describe every possible embodiment since describing every possible embodiment would be impractical, if not impossible. Numerous alternative embodiments could be implemented, using either current technology or technology developed after the filing date of this patent, which would still fall within the scope of the claims. To the extent that any term recited in the claims at the end of this patent is referred to in this patent in a manner consistent with a single meaning, that is done for sake of clarity only so as to not confuse the reader, and it is not intended that such claim term by limited, by implication or otherwise, to that single meaning.

Various embodiments of the invention are described herein and as depicted in the drawings. It is expressly understood that although some embodiments include compression bandages, pads, and securing fastener, and raised retention elements, the invention is not limited to these embodiments.

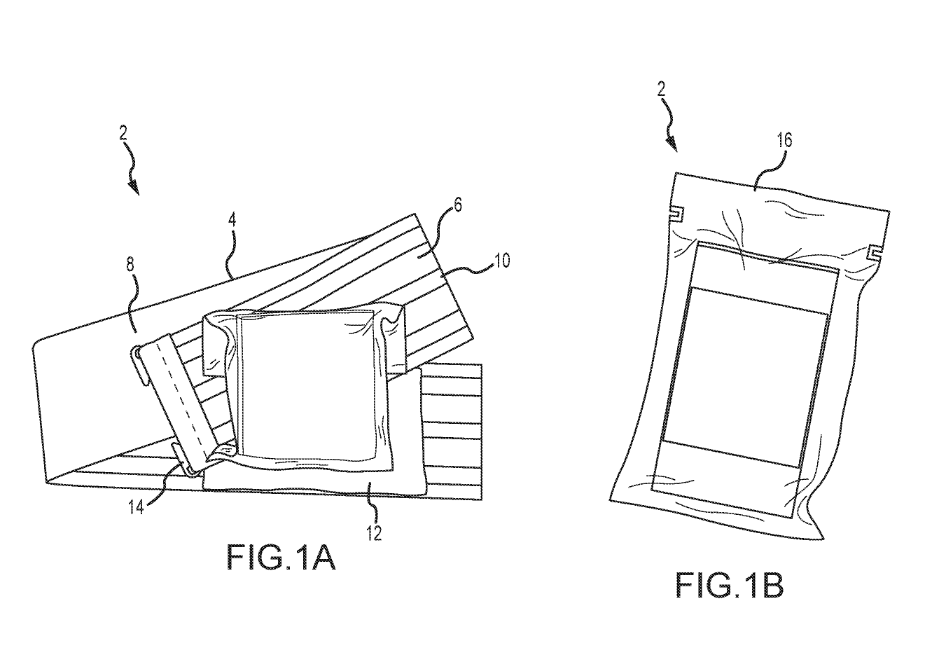

Now referring to FIG. 1A, a compression bandage 2 comprising a stretchable material 4 with a first side 6 and a second side 8 is provided. The stretchable material 4 has elastic properties that allow the stretchable material 4 to extend between a first length and a second length. The stretchable material 4 has the first length when the stretchable material 4 is in a relaxed state, meaning there are no external forces applied to the stretchable material 4. Conversely, the stretchable material 4 has the second length when the stretchable material 4 is in a tensioned state, meaning there are external forces applied to the stretchable material 4. In the embodiment in FIG. 1A, the second length is longer than the first length. This aspect of the stretchable material 4 allows a pad 12 to be placed over a wound, and then the stretchable material 4 may be stretched to the second length and wrapped upon itself to generate a compressive force on the pad 12 and the wound.

A securing fastener 14 and a raised retention element 10 may be used to help fix the stretchable material 4 in place once the stretchable material 4 is wrapped upon itself and providing a compressive force. The securing fastener 14 depicted in FIG. 1A is a clip disposed on one end of the stretchable material 4, wherein the end is wrapped around a body portion of the clip and secured, in this case stitched, onto the stretchable material 4 to hold the clip in place. The body portion of the clip extends further than the width of the stretchable material 4 in some embodiments. Clip ends are disposed at either end of the body portion of the clip, and the clip ends curl and extend towards the center of the stretchable material 4. Each clip end has a plurality of teeth on an inner surface that help grip the stretchable material 4 when the stretchable material 4 is wrapped upon itself to provide a compressive force. It will be appreciated that clips comprising a clip body and two clip ends are not the only securing fastener 14 contemplated by the invention. In other embodiments, the securing fastener 14 includes, but is not limited to, Velcro®, buttons, tape, snap fasteners, latches, magnets, adhesives, lugs, zippers, a force provided by a user, suction cup, electric inductions, etc.

Now referring to FIG. 1B, a compression bandage 2 stored in packaging 16 is provided. In this embodiment, the compressive bandage 2 is folded into a more compact arrangement, placed into the packaging 16, and then the packaging 16 is vacuum sealed to remove excess air. The resulting compression bandage 2 and packaging 16 is smaller in volume than the compression bandage 2 and packaging before the vacuum sealing process. In some exemplary embodiments, the volume dimensions of the packaging 16 may be 4″×4½″×¾″. In other exemplary embodiments, the volume dimensions of the packaging 16 may be 4″×6¾″×½″.

Now referring to FIG. 1C, an additional securing fastener 14 is provided. In this embodiment, the securing fastener comprises a bar that generally runs along the lateral width of the stretchable material 4. The end of the stretchable material 4 is wrapped around and stitched into place, but this end has a split or aperture in the stretchable material 4 that allows a protrusion to extend from the bar and through the stretchable material 4. This protrusion comprises four backward-facing hooks like a fishhook or arrowhead. Thus, when the securing fastener 14 is utilized, the hooks dig into the outer surface of the stretchable material 4 to secure the bandage 2 and provide a compressive force.

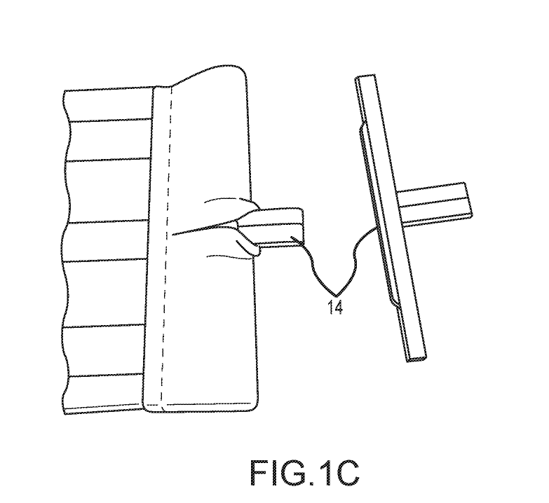

Now referring to FIG. 2, a top plan view of a compression bandage 2 is provided. This view of the compression bandage 2 illustrates the first side 6 or inner side of the stretchable material 4. The stretchable material 4 has a first end 18 and a second end 20, and a pad 12 is positioned proximate to the first end 18. The second end 20 of the stretchable material 4 is folded upon itself and stitched in place to accommodate a securing fastener 14 such as a clip.

Extending between the first and second ends 18, 20 of the stretchable material 4 are a first raised retention element 22, a second raised retention element 24, and a third raised retention element 26. The stretchable material 4 may have a first friction coefficient, and the raised retention elements 22, 24, 26 may have a second friction coefficient where the second friction coefficient is greater than the first friction coefficient. Thus, the raised retention elements 22, 24, 26 contribute to the ability of the stretchable material 4 to wrap upon itself and provide a compressive force.

The raised retention elements 22, 24, 26 extend between the first and second ends 18, 20 of the stretchable material 4. However, it will be appreciated that in other embodiments, the raised retention elements 22, 24, 26 may only extend a portion of the distance between the first and second ends 18, 20 of the stretchable material 4. In some embodiments, the raised retention elements 22, 24, 26 extend between approximately 95% and 60% of the distance between the first and second ends 18, 20 of the stretchable material 4, where the term “approximately” implies a variation of +/−10%. In various embodiments, the raised retention elements 22, 24, 26 extend between approximately 5% and 60% of the distance between the first and second ends 18, 20 of the stretchable material 4. Further, it will be appreciated that not all raised retention elements 22, 24, 26, if there are multiple raised retention elements, will extend the same distance between the first and second ends 18, 20 of the stretchable material 4.

Embodiments of the invention are not necessarily limited to the three parallel raised retention elements 22, 24, 26 depicted in FIG. 2. For example, the stretchable material 4 may have zero, one, two, four, five, etc. raised retention elements. Further, when there are multiple raised retention elements 22, 24, 26, these raised retention elements 22, 24, 26 may not be parallel to each other and spaced equidistant from each other. It will be appreciated that the multiple raised retention elements 22, 24, 26 may be spaced various distances from one another, and the multiple raised retention elements 22, 24, 26 may be oriented, for example, perpendicularly to one another.

The pad 12 in FIG. 2 is interconnected to the stretchable material 4 at a single, central location 28 to improve the absorbability of the pad 12 and to increase the ability of the pad 12 to stay positioned over the wound. Prior art bandages have pads that are typically interconnected to a strap or material around the entire perimeter of the pad. This type of interconnection compresses the pad along the perimeter of the pad which leaves only a small portion at the center of the pad that extends above the bandage. Having this much area of the pad compressed into the bandage reduces the ability of the pad to absorb fluids, including blood from a wound. This configuration also makes it more difficult to secure a clip or other securing fastener to the stretchable material 4.

In contrast, various embodiments of the invention include a pad 12 interconnected to a stretchable material 4 in the center of the pad. In FIG. 2, this interconnection is a stitched seam, which may be a small line or point. The pad 12 is compressed at a central point but not at a perimeter of the pad 12. This interconnection maximizes the pad 12 area that is not compressed, which increases the ability of the pad 12 to absorb fluid and allows a clip or other securing fastener to simply attach to the stretchable material 4 and not the stretchable material 4 in combination with the pad 12.

In addition, the central interconnection point between the pad 12 and the stretchable material 4 improves the ability of the pad to remain over a wound. Referring again to the prior art pad with a perimeter interconnection, these pads have a position and orientation that is identical to the stretchable material. This means that if the bandage stretchable material is not accurately placed over the wound with the proper orientation, then the pad may not effectively absorb fluid or apply a compressive force to the wound. In contrast, the central interconnection point of the embodiment in FIG. 2 allows the pad 12 to move from side-to-side to remain over the wound. The central interconnection point also allows the pad 12 to pivot or rotate to remain over the wound if the stretchable material covers the wound at an angle.

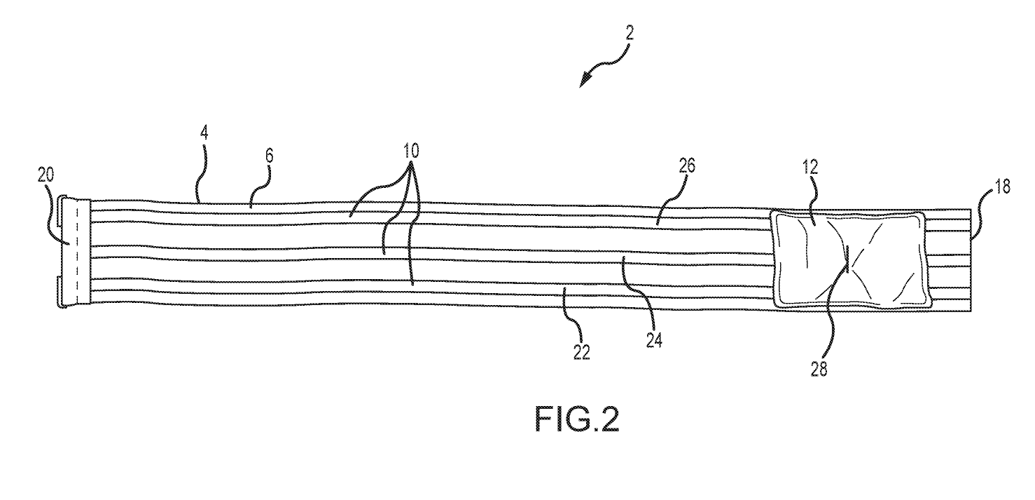

Now referring to FIG. 3, a detailed view of the raised retention element 10 and the stretchable material 4 is provided. The stretchable material 4 is made from a particular arrangement of threads, which are typically referred to as warp and weft. Warp refers to the thread is oriented in the longitudinal direction of the stretchable material 4, and the weft is oriented in the lateral direction of the stretchable material 4. The raised retention element 10 shown in FIG. 3 is oriented with the warp in the longitudinal direction of the stretchable material 4. Portions of the weft thread secure the raised retention element 20 to the stretchable material 4. The raised retention element 10 shown in FIG. 3 is comprised of seven separate strands 30 of material oriented parallel to each other. It will be appreciated that other embodiment of the invention may have other numbers of strands 30 including zero, one, two, three, four, five, six, eight, etc.

A strand angle 32 between the individual strand 30 and a longitudinal plane of the stretchable material 4 is also shown in FIG. 3. The strand angle 32 continuously changes as the strand 30 move along the stretchable material 4 in the longitudinal direction. Therefore, the strand angle 32 may be expressed as a function of the position of the stand 30 along the longitudinal length of the stretchable material 4, for example, as a sine function. The strand angle 32 in FIG. 3 varies between approximately +30° and −30°. The strand angle 32 may be described by other functions or shapes as the strand 30 moves along the stretchable material 4. For example, sawtooth waves, triangle waves, and square waves may be used to describe the strand angle 32. In other embodiments, the strand angle 32 does not vary, which means that the strand 30 extends straight in one direction. In various embodiments, the strand 30 may be oriented in the warp direction, or the strand 30 may be oriented in the weft direction. In yet other alternative embodiments, the strand 30 may be oriented between the warp and weft directions.

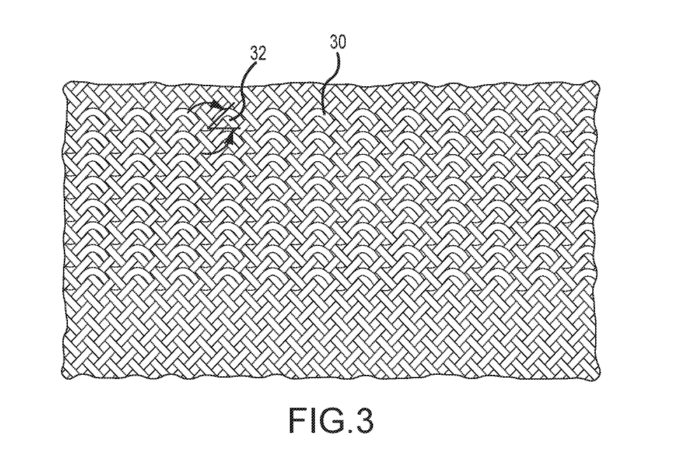

Now referring to FIG. 4, a compression bandage 2 having a particular strand 30 configuration is provided. In the embodiment described in FIG. 3, the strands 30 have a sinusoidal shape, and the strands 30 are positioned such that the strands 30 align with each other. Put another way, the sinusoidal shapes of the strands 30 are in phase with one another. The strands 30 in FIG. 4 are not aligned; the strands 30 are offset. Using the sinusoidal analogy, the two strands 30 in FIG. 4 are out of phase by 180 degrees, meaning the crest of one strand is adjacent to the trough of the other strand.

Various strand offsets in the warp direction of the bandage 2 may help provide additional friction between the raised retention element and the stretchable material of the bandage 2. For example, the 180 degree offset in FIG. 4 concentrates the strand material in some areas, which can help the raised retention element press into the stretchable material of the bandage.

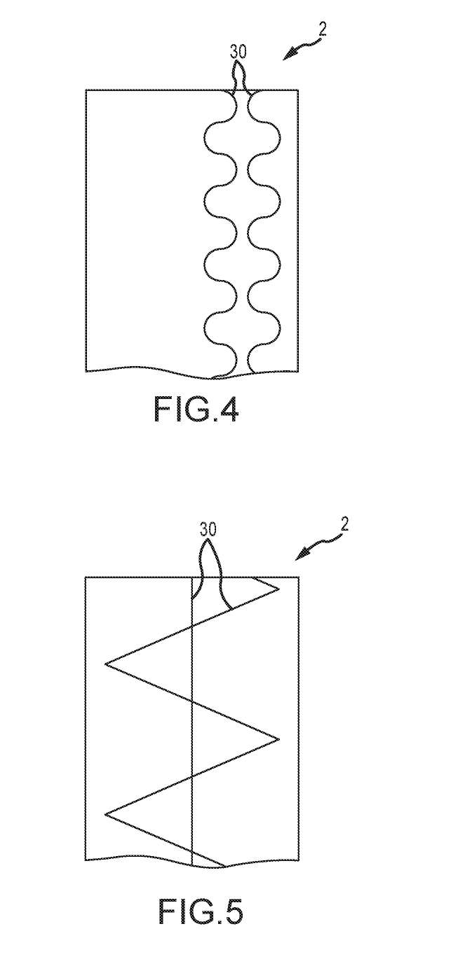

Now referring to FIG. 5, a compression bandage 2 having another particular strand 30 configuration is provided. In this embodiment, one strand 30 is a triangular wave shape that zigzags across the entire width of the stretchable material of the bandage 2. This may improve the ability of the bandage 2 to wrap upon itself because more area is covered by the strand 30. The other stand 30 is straight and aligned in the warp direction of the stretchable material of the bandage. As such, the two differently shaped strands 30 overlap each other periodically along the warp direction of the stretchable material of the bandage. The overlap may be the straight strand 30 over the triangular strand 30, or vice versa. Further, the strands 30 may alternate overlaps or overlap each other according to no particular sequence. The overlapping strands 30 create a point where the griping feature is raised above other features on a first or inner side of the stretchable material of the bandage 2. Thus, the overlap allows a portion of the raised retention element to embed itself in the second or outer side of the bandage when the bandage 2 is wrapped over a wound to provide a compressive force.

In a further embodiment of the invention, the stretchable material of the bandage may also have one or more raised retention elements disposed on its second or outer surface. Therefore, when the bandage 2 is wrapped upon itself to provide a compressive force, the first set of raised retention elements contacts a second set of raised retention elements on the second or outer surface of the stretchable material rather than the stretchable material itself. This may be advantageous when the coefficient of friction for the raised retention elements is greater than the coefficient of friction for the stretchable material of the bandage 2. While in some embodiments the coefficient of friction for the two sets of raised retention elements may be the same, it will be appreciated that in some embodiments the first and second sets of raised retention elements may have different coefficients of friction.

In addition, the particular configuration of the two sets of raised retention elements and their constituent strands may provide physical features that interlock with each other in addition to reliance on a relatively high coefficient of friction. For example, the triangular wave strand 30 in FIG. 5 may be used on both sides of the stretchable material of the bandage 2. Thus, when the bandage 2 is wrapped upon itself, the triangular wave shapes of both sets of raised retention elements may interlock with each other. Stated another way, the raised retention elements may extend from the stretchable material to increase the surface area of the raised retention elements that contact the stretchable material. This aspect also increases the number of instances of static friction coefficients, which are generally higher than dynamic friction coefficients.

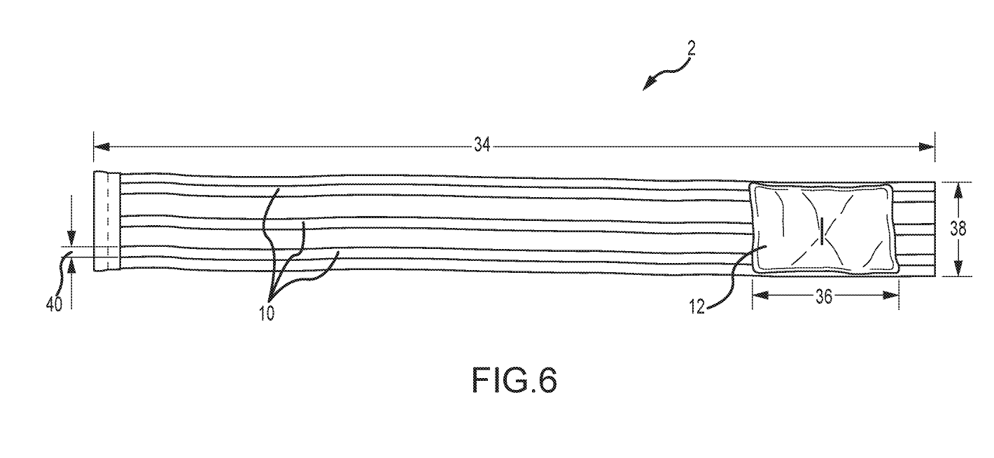

Now referring to FIG. 6, various dimensions of an exemplary compression bandage 2 are provided. First, the bandage 2 has an overall length 34. In some embodiments, the overall length 34 is between approximately 12″ and 60″. In various embodiments, the overall length 34 is between approximately 24″ and 48″. In certain embodiments, the overall length is approximately 36″. Similarly, the pad 12 may also comprise a pad length 36. In some embodiments, the pad length 36 is between approximately 2″ and 24″. In various embodiments, the pad length 36 is between approximately 4″ and 12″. In certain embodiments, the pad length 36 is approximately 6″.

The bandage 2, specifically the stretchable material 4, in FIG. 6 and the pad 12 may share the same width dimension 38. In some embodiments, the width dimension 38 is between approximately 2″ and 12″. In various embodiments, the width dimension 38 is between approximately 3″ and 8″. In certain embodiments, the width dimension 38 is approximately 4″. It will be appreciated that the stretchable material 4 and the pad 12 do not necessarily share the same width dimension 38 in all embodiments of the invention.

The raised retention elements 10 may also have a gripping width 40. In some embodiments, the gripping width 40 is between approximately ⅛″ and 2″. In various embodiments, the gripping width 40 is between approximately ¼″ and 1″. In certain embodiments, the gripping width 40 is approximately ½″.

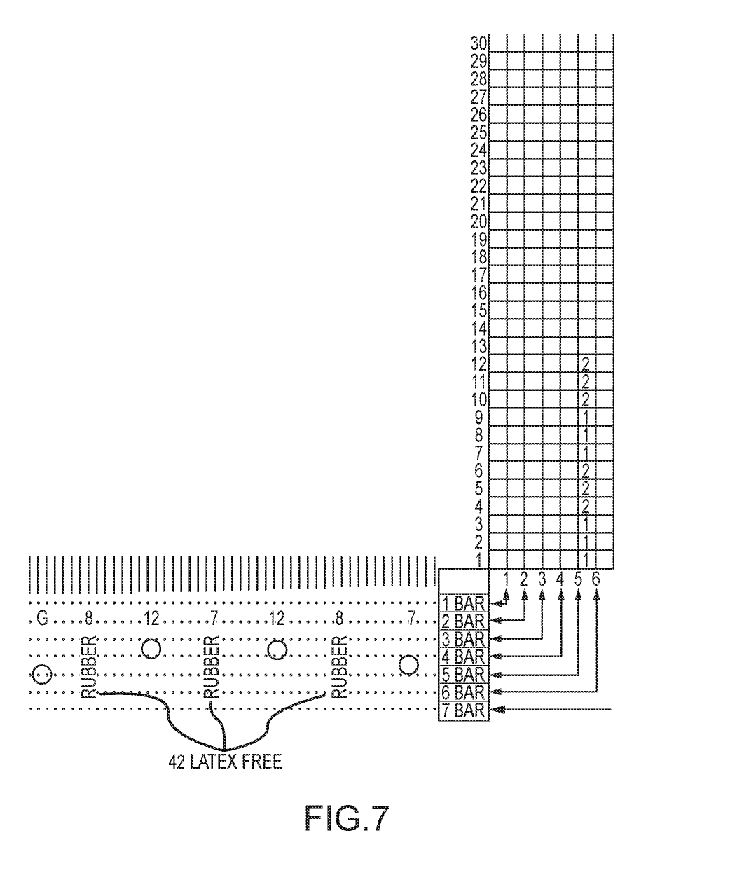

Now referring to FIG. 7, a diagram of a warp and weft order is provided. Among other information provided in this diagram, the material of the strands in this embodiment is latex-free rubber. The coefficient of friction for rubber is greater against canvas or cotton than canvas or cotton against itself.

It will be appreciated that embodiments of the invention may incorporate additional elements such as hooks, H-bars, and cups. A hook may be disposed on the second or outer surface of the stretchable material. When a user, most likely a medic or other emergency personnel, is applying the bandage to a wound, the user may thread the stretchable material through the hook and reverse the direction of the stretchable material. The hook and the direction reversal allow a user to pull the stretchable material and pad tight over the wound before the user completes wrapping the stretchable material upon itself and securing the stretchable material in place. An H-Bar is a similar feature disposed on the second or outer surface of the stretchable material. Instead of threading the stretchable material through a hook, a user wraps the thread around one arm of the “H” to reverse the direction of the stretchable material. Lastly, a cup may be disposed in or on a pad where the curved surface of the cup is directed downward toward the wound. Incorporating the cup allows more force to be concentrated in a small area over the wound, which help stem bleeding more effectively in some instances.

The invention has significant benefits across a broad spectrum of endeavors. It is the Applicant's intent that this specification and the claims appended hereto be accorded a breadth in keeping with the scope and spirit of the invention being disclosed despite what might appear to be limiting language imposed by the requirements of referring to the specific examples disclosed.

The phrases “at least one”, “one or more”, and “and/or”, as used herein, are open-ended expressions that are both conjunctive and disjunctive in operation. For example, each of the expressions “at least one of A, B, and C”, “at least one of A, B, or C”, “one or more of A, B, and C”, “one or more of A, B, or C,” and “A, B, and/or C” means A alone, B alone, C alone, A and B together, A and C together, B and C together, or A, B, and C together.

Unless otherwise indicated, all numbers expressing quantities, dimensions, conditions, and so forth used in the specification, drawings, and claims are to be understood as being modified in all instances by the term “about.”

The term “a” or “an” entity, as used herein, refers to one or more of that entity. As such, the terms “a” (or “an”), “one or more” and “at least one” can be used interchangeably herein.

The use of “including,” “comprising,” or “having,” and variations thereof, is meant to encompass the items listed thereafter and equivalents thereof as well as additional items. Accordingly, the terms “including,” “comprising,” or “having” and variations thereof can be used interchangeably herein.

It shall be understood that the term “means” as used herein shall be given its broadest possible interpretation in accordance with 35 U.S.C. §112(f). Accordingly, a claim incorporating the term “means” shall cover all structures, materials, or acts set forth herein, and all of the equivalents thereof. Further, the structures, materials, or acts, and the equivalents thereof, shall include all those described in the summary of the invention, brief description of the drawings, detailed description, abstract, and claims themselves. The foregoing description of the invention has been presented for illustration and description purposes. However, the description is not intended to limit the invention to only the forms disclosed herein. In the foregoing Detailed Description for example, various features of the invention are grouped together in one or more embodiments for the purpose of streamlining the disclosure. This method of disclosure is not to be interpreted as reflecting an intention that the claimed invention requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the invention.

Consequently, variations and modifications commensurate with the above teachings and skill and knowledge of the relevant art are within the scope of the invention. The embodiments described herein above are further intended to explain best modes of practicing the invention and to enable others skilled in the art to utilize the invention in such a manner, or include other embodiments with various modifications as required by the particular application(s) or use(s) of the invention. Thus, it is intended that the claims be construed to include alternative embodiments to the extent permitted by the prior art.

What is claimed is:

1. A compression bandage for treating an injury, comprising:
a stretchable material having a first end, a second end, and a first length between said first and said second ends when said stretchable material is in a relaxed state, said stretchable material having a second length between said first and second ends when said stretchable material is in a tensioned state, said second length being longer than said first length, said stretchable material also having a first side, a second side, and a first friction coefficient;
a retention element disposed on said first side of said stretchable material, and said retention element raised above said first side, said raised retention element having a second friction coefficient, said second friction coefficient being greater than said first friction coefficient;
a pad disposed on said first side of said stretchable material; and
a securing fastener positioned proximate to said second end of said stretchable material, wherein said securing fastener is configured to hold said second end of said stretchable material in said tensioned state against said second side of said stretchable material such that said raised retention element contacts said second side of said stretchable material and prevents said second end of said stretchable material from slipping relative to said second side of said stretchable material.

2. The compression bandage of claim 1, wherein said securing fastener is one of a hook and loop fastener, a button, a tape, a snap fastener, a latch, a magnet, an adhesive, a lug, a zipper, a force provided by a user, a suction cup, an electric induction, and a clip.

3. The compression bandage of claim 1, wherein said raised retention element longitudinally extends between said first end and said second end of said stretchable material.

4. The compression bandage of claim 1, further comprising:
a second retention element disposed on said first side of said stretchable material, said second retention element raised above said first side, and said second raised retention element also having said second friction coefficient.

5. The compression bandage of claim 4, wherein said raised retention elements longitudinally extend between said first end and said second end of said stretchable material, and said raised retention elements are oriented substantially parallel to each other.

6. The compression bandage of claim 1, wherein said pad is interconnected to said stretchable material at a central location of said pad.

7. The compression bandage of claim 1, wherein said pad is positioned proximate to said first end of said stretchable material.

8. The compression bandage of claim 1, wherein said raised retention element comprises a plurality of strands, and wherein each strand forms a non-zero angle with a longitudinal plane extending from said first end to said second end of said stretchable material.

9. The compression bandage of claim 1, wherein said raised retention element comprises a plurality of strands, wherein each strand forms a sinusoidal pattern as each strand extends from said first end to said second end of said stretchable material.

10. A method for securing a bandage to an appendage of a person, comprising:
providing a stretchable material having a first end, a second end, a first side, a second side, and a first friction coefficient;
providing a retention element on said first side of said stretchable material, said retention element raised above said first side, said raised retention element having a second friction coefficient, and said second friction coefficient being greater than said first friction coefficient;
providing a pad on said first side of said stretchable material;
locating said pad on an appendage of a person;
wrapping said stretchable material around said appendage such that at least a portion of said raised retention element contacts said second side of said stretchable material to induce a friction force; and
securing said portion of said raised retention element against said second side of said stretchable material with a securing fastener.

11. The method of claim 10, further comprising:
stretching said stretchable material between a first length and a second length to wrap said stretchable material around said appendage, wherein said second length is longer than said first length.

12. The method of claim 10, further comprising:
providing said securing fastener, wherein said securing fastener is one of a hook and loop fastener, a button, a tape, a snap fastener, a latch, a magnet, an adhesive, a lug, a zipper, a force provided by a user, a suction cup, an electric induction, and a clip.

13. The method of claim 10, wherein said raised retention element longitudinally extends between said first end and said second end of said stretchable material.

14. The method of claim 10, further comprising:
providing a second retention element on said first side of said stretchable material, said second retention element raised above said first side, and said second raised retention element also having said second friction coefficient.

15. The method of claim 14, wherein said raised retention elements longitudinally extend between said first end and said second end of said stretchable material, and said raised retention elements are oriented substantially parallel to each other.

16. The method of claim 10, further comprising interconnecting said pad to said stretchable material at a central location of said pad.

17. The method of claim 10, further comprising positioning said pad proximate to said first end of said stretchable material.

18. The method of claim 10, wherein said raised retention element comprises a plurality of strands, and wherein each strand forms a non-zero angle with a longitudinal plane extending from said first end to said second end of said stretchable material.

19. The method of claim 10, further comprising providing said raised retention elements, wherein said raised retention element comprises a plurality of strands, wherein each strand forms a sinusoidal pattern as each strand extends from said first end to said second end of said stretchable material.

20. A compression bandage system for treating an injury, comprising:
a stretchable material having a first end, a second end, and a first length between said first and said second ends when said stretchable material is in a relaxed state, said stretchable material having a second length between said first and second ends when said stretchable material is in a tensioned state, said second length being longer than said first length, said stretchable material also having a first side, a second side, and a first friction coefficient;
a plurality of raised retention elements disposed on said first side of said stretchable material, wherein said raised retention elements longitudinally extend between said first end and said second end of said stretchable material, and said raised retention elements are oriented substantially parallel to each other, wherein said raised retention elements each comprise a plurality of strands, wherein each strand forms a sinusoidal pattern as each strand extends from said first end to said second end of said stretchable material, said raised retention elements having a second friction coefficient, said second friction coefficient being greater than said first friction coefficient;
a pad disposed on said first side of said stretchable material at a central location of said pad, and said pad proximate to said first end of said stretchable material; and
a securing fastener positioned proximate to said second end of said stretchable material, wherein said securing fastener holds said second end of said stretchable material in place relative to said second side of said stretchable material such that said plurality of raised retention elements contacts said second side of said stretchable material and prevents said second end of said stretchable material from slipping relative to said second side of said stretchable material.
